(19) United States
(12) Patent Application Publication
(10) Pub. No.: US 2026/0060952 A1
(43) Pub. Date: Mar. 5, 2026

(54) Prophylactic, Progression Inhibitor, Ameliorant for Visual Field Defect Disorders, Light-Induced Ocular Tissue Disorders and Related Disorders, and Food Product

(71) Applicant: REFINE HOLDINGS CO., LTD., Gifu (JP)

(72) Inventors: Makoto TSUBOI, Tokyo (JP); Yasuko SAKATA, Tokyo (JP)

(73) Assignee: REFINE HOLDINGS CO., LTD., Gifu (JP)

(21) Appl. No.: 19/116,526

(22) PCT Filed: Sep. 28, 2023
(86) PCT No.: PCT/JP2023/035386

§ 371 Date: Apr. 15, 2025

(30) Foreign Application Priority Data
Oct. 2, 2022 (JP) 2022-159173
May 30, 2023 (JP) 2023-089104

(51) Int. Cl. A61K 31/20; A61K 36/02; A61P 27/06

(52) U.S. Cl. CPC A61K 31/20; A61K 36/02; A61P 27/06; A61K 2236/35

(57) ABSTRACT

Provided is a composition for visual field defect disorders, glaucoma, light-induced ocular tissue damage and related disorders, which can be used as a food or drug for long-term administration, prevention, symptom relief and improvement. A composition comprising a triglyceride represented by the formula (I):

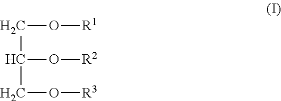

(wherein R¹, R², and R³ are each a saturated fatty acid residue, and at least one of them is a pentadecanoic acid residue) as an active ingredient.

### TECHNICAL FIELD

The present technology relates to a composition that ameliorates ocular tissue disorders caused by endoplasmic reticulum stress in the retina due to light stimuli such as glaucoma, LED, or blue light, which narrow or obscure the visual field (visible range) due to damage to the optic nerve for any reason, and prevents, prophylaxes, and ameliorates systemic symptoms affected by ocular tissue disorders and visual function decline.

### BACKGROUND ART

Endoplasmic reticulum stress refers to a state in which cells are exposed to various internal or external environmental changes, resulting in an abnormal expression of the protein synthesis system in the endoplasmic reticulum lumen, increasing the protein amount, accumulating proteins that are not normally eliminated, and accumulating proteins as defective proteins because they are not properly folded. Known factors that cause endoplasmic reticulum stress include nutritional starvation, intracellular calcium concentration disturbance, hypoxia, expression of mutant proteins, and viral infection. When cells are under endoplasmic reticulum stress, they activate lipid synthesis and expand the endoplasmic reticulum to increase protein folding processing capacity in order to maintain homeostasis. In the relatively mild stage of defective protein accumulation, an endoplasmic reticulum stress response mechanism for defective protein elimination is performed. However, if the stress state is severe or lasts for a long period of time, denatured proteins accumulate in the endoplasmic reticulum. This causes adverse effects on cells. To avoid endoplasmic reticulum stress-induced damage and maintain homeostasis, cells induce cell death (apoptosis) in systemic tissues and organs. When this reaction occurs in neural tissue, degeneration and loss of nerve fibers (neurons) occur. It has been suggested that endoplasmic reticulum stress is involved in the onset of neurodegenerative diseases (see Non-Patent Literature 1).

Glaucoma is a progressive multifactorial neurological disease caused by retinal ganglion cell death, and irreversible retinal ganglion cell death results in visual field defects accompanied by decreased contrast sensitivity. In addition to elevated intraocular pressure, genetic predisposition and environmental factors, endoplasmic reticulum stress is attracting attention as a mechanism that causes cell death in retinal ganglion cell death (see Non-Patent Literature 2). In addition, although the retina is an important organ responsible for visual reception, there are concerns about the effects of light exposure on the retina. It has been suggested that blue LED light irradiation causes photoreceptor cell death, and endoplasmic reticulum stress is involved in the mechanism (see Non-Patent Literature 2). That is, excessive exposure of cells to blue LED light, together with increased production of reactive oxygen species, causes endoplasmic reticulum stress, especially increased expression cell death and of Activating Transcriptional Factor 4 (ATF4), a downstream factor of the PERK pathway.

Conventionally, a composition containing hesperidin as an active ingredient has been proposed as a composition for inhibiting retinal ganglion cell death in glaucoma and preventing and treating glaucoma (Patent Literatures 1 and 2). Furthermore, a composition containing lactic acid bacteria, represented by Lactobacillus paracasei KW3110 strain, has been proposed as a composition for preventing visual dysfunction caused by exposure to light such as blue light (Patent Literature 3). It is argued that intake of Lactobacillus paracasei KW3110 strain antagonizes retinal cell death caused by blue light irradiation, maintains significantly thicker retinal thickness, and alleviates eyestrain, but the extent of its effect is not sufficient, and the mechanism of action, such as inhibiting cell death, has not been particularly clarified. In addition to this, various substances such as taurooursodeoxycholic acid (TUDCA), blueberry extract, and anthocyanin have been disclosed as being effective against ocular tissue disorders, but the extent of their effects was not fully satisfactory.

### PRIOR ART DOCUMENTS

### Non-Patent Literatures

[Non-Patent Literature 1] Satoshi Kanemoto, Kazunori Imaizumi, Endoplasmic Reticulum Stress and Diseases, Journal of Japanese Biochemical Society 90 (1): 51-59 (2018)
[Non-Patent Literature 2] Masamitsu Shimazawa, Hideaki Hara, Neuro-Ophthalmology 2015 Vol. 32 No. 3, p. 240-247, Pathophysiology of Retina-Optic Nerve Degeneration and its Therapeutic Strategies for Neuroprotection
[Non-Patent Literature 3] Yoshiki K et al, Sci. Rep. 2014

### PATENT LITERATURES

[Patent Literature 1] International Publication No. WO2017/010520
[Patent Literature 2] JP 2021-080259 A
[Patent Literature 3] JP 2020-63297 A

### SUMMARY OF THE INVENTION

### Problem to be Solved by the Invention

As described above, conventionally, there is no known orally ingestible composition that ameliorates ocular tissue disorders caused by endoplasmic reticulum stress in the retina due to light stimuli such as LED and blue light, and effectively prevents, prophylaxes, and ameliorates systemic symptoms affected by ocular tissue disorders and visual function decline, and there is a demand for food and drugs that can be taken over a long period of time and effectively prevent, alleviate symptoms, and ameliorate them. Furthermore, there was no known orally ingestible composition that can inhibit retinal ganglion cell death and effectively prevent and treat visual field defect disorders or glaucoma. Therefore, the problem to be solved by the present technology is to improve the state of endoplasmic reticulum stress in the retina induced by light exposure such as blue light, thereby ameliorating functional decline and disorders in ocular tissues, and further effectively preventing, prophylaxes, and ameliorating systemic symptoms affected by ocular tissue disorders and visual function decline, and to provide a prophylactic, progression inhibitor, ameliorant for light-induced ocular tissue disorders and related disorders, and food products that can be used as food and drugs. The problem to be solved by the present technology is also to provide a prophylactic, progression inhibitor, ameliorant for visual field defect disorders, glaucoma, and related disorders, and food products that can inhibit retinal ganglion cell death and effectively prevent and treat visual field defect disorders and glaucoma.

### Means for Solving the Problem

As a result of intensive studies to solve the above problems, the present inventors have found that triglycerides composed of saturated fatty acids mainly containing pentadecanoic acid (C15) (pentadecanoic acid triglyceride: hereinafter sometimes referred to as “PdATG”) inhibit the accumulation of denatured proteins in the endoplasmic reticulum caused by endoplasmic reticulum stress induced by excessive light exposure, and reduce cell death (apoptosis) caused as a result, thereby ameliorating functional decline and disorders in ocular tissues, and further effectively preventing, prophylaxes, and ameliorating systemic symptoms affected by ocular tissue disorders and visual function decline, thereby completing the present technology.

A prophylactic for light-induced ocular tissue disorders and related disorders according to the first aspect of the present technology that solves the above problems contains a triglyceride represented by the following formula (I) as an active ingredient:

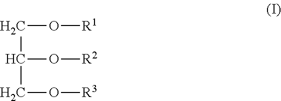

(In the formula, R¹, R² and R³ are each saturated fatty acid residues, and at least one of them is a pentadecanoic acid residue.)

In one embodiment of the prophylactic for light-induced ocular tissue disorders and related disorders, the triglyceride of formula (I) preferably has R¹ and R² or R¹ and R³ as pentadecanoic acid residues. In another embodiment, any one of R¹, R² and R³ may be tridecylic acid (C13), myristic acid residue (C14), palmitic acid residue (C16) or margaric acid residue (C17).

In another preferred embodiment, it may contain the triglyceride of the above formula (I) in which all of R¹, R² and R³ are pentadecanoic acid residues, and the triglyceride of formula (I) in which any two of R¹, R² and R³ are pentadecanoic acid residues and the other one is myristic acid or palmitic acid residue.

In still another preferred embodiment of the prophylactic for light-induced ocular tissue disorders and related disorders of the present technology, the triglyceride of formula (I) is derived from algae of the genus Aurantiochytrium or Schizochytrium, and in the formula, R¹, R² and R³ are each saturated fatty acid residues, and at least one of them may be a triglyceride that is a pentadecanoic acid residue. Furthermore, it may be a mixture containing unsaturated fatty acids derived from algae of the genus Aurantiochytrium or Schizochytrium.

In the second aspect of the present technology, there is provided a progression inhibitor for light-induced ocular tissue disorders and related disorders containing the triglyceride represented by the above formula (I) as an active ingredient.

In the third aspect of the present technology, there is provided an ameliorant for light-induced ocular tissue disorders and related disorders containing the triglyceride represented by the above formula (I) as an active ingredient.

Furthermore, in the fourth aspect of the present technology, there is provided a food product containing the triglyceride represented by the above formula (I) as an active ingredient. This food product is preferably used, for example, as a health food, a food with function claims, or a food for specified health use, and for food improvement.

A prophylactic for visual field defect disorders, glaucoma, and related disorders according to the fifth aspect of the present technology that solves the above problems contains the triglyceride represented by the above formula (I) as an active ingredient.

In one embodiment of the prophylactic for visual field defect disorders, glaucoma, and related disorders, the triglyceride of formula (I) preferably has R¹ and R² or R¹ and R³ as pentadecanoic acid residues. In another embodiment, any one of R¹, R² and R³ may be tridecylic acid (C13), myristic acid residue (C14), palmitic acid residue (C16) or margaric acid residue (C17).

In another preferred embodiment, it may contain the triglyceride of the above formula (I) in which all of R¹, R² and R³ are pentadecanoic acid residues, and the triglyceride of formula (I) in which any two of R¹, R² and R³ are pentadecanoic acid residues and the other one is myristic acid or palmitic acid residue.

In still another preferred embodiment of the prophylactic for visual field defect disorders, glaucoma, and related disorders of the present technology, the triglyceride of formula (I) is derived from algae of the genus Aurantiochytrium or Schizochytrium, and in the formula, R¹, R² and R³ are each saturated fatty acid residues, and at least one of them may be a triglyceride that is a pentadecanoic acid residue. Furthermore, it may be a mixture containing unsaturated fatty acids derived from algae of the genus Aurantiochytrium or Schizochytrium.

In the sixth aspect of the present technology, there is provided a progression inhibitor for visual field defect disorders, glaucoma, and related disorders containing the triglyceride represented by the above formula (I) as an active ingredient.

In the seventh aspect of the present technology, there is provided an ameliorant for visual field defect disorders, glaucoma, and related disorders containing the triglyceride represented by the above formula (I) as an active ingredient.

### Effects of the Invention

According to the composition containing the triglyceride of the present technology, endoplasmic reticulum stress can be inhibited in the retinal tissue of mammalian cells, and food and drugs for prevention, symptom alleviation, and amelioration that can be taken over a long period of time can be provided.

### BRIEF DESCRIPTION OF THE DRAWINGS

FIG. 1 is a diagram showing the experimental details of examining the inhibitory effect on cell death by the added drug in retinal cells after inducing disorders, where A is the experimental protocol, B is the result of Hoechst PI staining showing the change in cell rate with the added drug in endoplasmic reticulum stress induced by the addition of thapsigargin, C is a graph showing the relationship between the added amount of each drug and the dead cell rate, D is the result of Hoechst PI staining showing the change in cell rate with the added drug after inducing disorders by blue LED irradiation, and E is a graph showing the relationship between the added amount of each drug and the dead cell rate, in an example of the present technology.

FIG. 2 is a diagram showing the experimental details of examining the inhibitory effect of the added drug on the expression of endoplasmic reticulum stress-related proteins in retinal cells after inducing disorders, where A is the experimental protocol, B is the result of Western blotting evaluating the expression level of ATF4, an endoplasmic reticulum stress marker in endoplasmic reticulum stress induced by the addition of thapsigargin, C is a graph showing the relationship between the added amount of the drug and the expression level of ATF4, D is the result of Western blotting evaluating the expression level of ATF4, an endoplasmic reticulum stress marker in the added drug after inducing disorders by blue LED irradiation, and E is a graph showing the relationship between the added amount of each drug and the dead cell rate, in another example of the present technology.

FIG. 3 is a drawing showing a chart used when performing a visual field check in an example of the present technology.

FIG. 4 is a diagram showing a change in the visual field state before and after taking a drug of a glaucoma patient in an example of the present technology.

FIG. 5 is a diagram showing a change in the visual field state before and after instillation of a drug of a glaucoma patient in an example of the present technology.

### MODE FOR CARRYING OUT THE INVENTION

Next, the present technology will be described in more detail based on embodiments. It should be noted that the following embodiments do not limit the invention according to the claims, and all elements and combinations thereof described in each embodiment are not necessarily essential to the solution means of the present technology.

(Active ingredient) In the present specification, PdATG means an ester of glycerol with at least one pentadecanoic acid, and at least one, preferably any two, of R¹, R² and R³ shown in the following formula (I), for example, R¹ and R² or R¹ and R³, and more preferably all three of R¹, R² and R³, contain triglycerides that are pentadecanoic acid residues. The binding position of pentadecanoic acid to glyceride may be any of 1 to 3 positions.

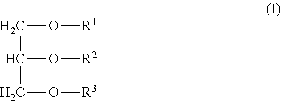

(In the formula, R¹, R² and R³ are each saturated fatty acid residues, and at least one of them is a pentadecanoic acid residue.)

In the formula, any one of the residues represented by R¹, R² and R³ may be a saturated fatty acid residue other than a pentadecanoic acid residue. The “saturated fatty acid” is a general term for fatty acids that do not have double bonds or triple bonds in the molecule, and is represented by the chemical formula CnH2n+1COOH. The saturated fatty acid is a linear or branched saturated fatty acid, and examples thereof include linear saturated fatty acids such as capric acid (C10), lauric acid (C12), tridecylic acid (C13), myristic acid (C14), pentadecanoic acid (C15), palmitic acid (C16), margaric acid (C17), stearic acid (C18), arachidic acid (C20), behenic acid (C22), lignoceric acid (C24) and cerotic acid (C26), and branched saturated fatty acids such as 2-hexyldecanoic acid (C16), 13-methylpentadecanoic acid (C16) and 16-methylheptadecanoic acid (C18).

The PdATG in a preferred embodiment contains both the triglyceride of the above formula (I) in which all of R¹, R² and R³ are pentadecanoic acid residues, and the triglyceride in which any two of R¹, R² and R³ are pentadecanoic acid residues and the other one is myristic acid or palmitic acid residue. The content ratio of both in this mixture is not particularly limited, but is preferably 1:2 to 2:1 by mass ratio, and more preferably about 1:1. Furthermore, each of them is contained in an amount of 10% by mass or more, preferably 20% by mass or more, based on the total amount of triglycerides. Furthermore, it is more preferable that the mixture of triglycerides containing two or more pentadecanoic acid residues is contained in 50% by mass or more in the oil and fat.

In a more preferred embodiment, PdATG is represented by the following formula (II) or (III).

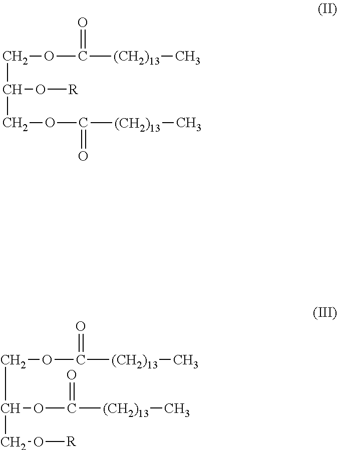

(However, in the above formulas (II) and (III), R is a C14-C16 saturated fatty acid.) It is more preferable that the mixture of triglycerides containing two or more pentadecanoic acid residues is contained in 50% by mass or more in the oil and fat, but even if the content of triglycerides containing two or more pentadecanoic acid residues is 50% by mass or less, the object can be achieved by increasing the intake. Therefore, the active ingredient of the present technology may be present in the form of a mixture of triglycerides containing two or more pentadecanoic acid residues, and can exhibit a function as an active ingredient by itself if it is contained at a purity of at least 1% by mass, preferably 50% by mass or more, more preferably 90% by mass or more, based on the total amount of triglycerides.

The active ingredient of the present technology may be present in a mixture with triglycerides other than the compound of formula (I), and can exhibit a function as an active ingredient by itself if it is contained at a purity of at least 1% by mass, preferably 50% by mass or more, more preferably 90% by mass or more, based on the total amount of triglycerides.

It is considered that the active ingredient of the present technology, which has at least one, preferably two or more, fatty such odd-chain acids, particularly pentadecanoic acid, in the molecule, exerts an effect of inhibiting abnormal proteins that accumulate in the endoplasmic reticulum due to endoplasmic reticulum stress in retinal cells, which will be described later, reducing cell death, and leading to normal.

(Method for Producing Triglyceride Mixture) The triglyceride mixture that is the active ingredient of the present technology may be chemically synthesized or naturally occurring. When it is natural, its source is not particularly limited. Examples include lipids produced by organisms in the body, such as livestock and poultry fats, fish and shellfish oils, vegetable oils, or lipid-producing microorganisms. From the viewpoint of industrial productivity, microorganisms such as algae, bacteria, fungi (including yeast), and/or protozoa are preferred. Preferred microorganisms include those selected from the group consisting of golden algae (microorganisms of the Stramenopiles kingdom, etc.), green algae, diatoms, dinoflagellates, yeast, and fungi of the genera Mucor and Mortierella. Members of the Stramenopiles microbial group include microalgae. Microalgae refer to organisms that perform photosynthesis that generates oxygen, excluding moss plants, ferns, and seed plants, and have a cell size of 1 μm to 100 μm in diameter. Labyrinthulomycetes, which are protozoa closely related to microalgae, are also included. Labyrinthulomycetes are heterotrophic marine eukaryotic microorganisms that do not perform photosynthesis and are widely distributed mainly in subtropical and tropical regions. Generally, Labyrinthulomycetes are broadly divided into the Labyrinthulaceae and family the family Thraustochytriaceae, and include the genera Labyrinthula, Aurantiochytrium, Schizochytrium, Thraustochytrium, Aplanochytrium, oblongichytrium, Botryochytrium, and Japonochytrium.

Aurantiochytrium, Schizochytrium, or Thraustochytrium are more preferable as Labyrinthulomycetes to be cultured. These species have relatively high production capacity of lipids and the like, and can produce hydrocarbons such as squalene, and are therefore suitably used for edible purposes, raw materials for biofuels, and the like.

The Labyrinthulomycetes may be cultured by any of batch culture, continuous culture, fed-batch culture, and other culture methods. The Labyrinthulomycetes can be cultured by any appropriate culture method such as shaking culture, aeration culture, aeration stirring culture, air-lift culture, and static culture. Among these culture methods, aeration stirring culture or air-lift culture is more preferable. As culture apparatus used for culturing Labyrinthulomycetes, for example, a mechanical stirring reactor, an air-lift reactor, a packed bed reactor, a fluidized bed reactor, or the like can be used. As a culture container, various containers such as a tank, a jar fermenter, a flask, a dish, a culture bag, a tube, and a test tube can be used according to the purpose of culture, culture capacity, and the like. The culture container may be made of any appropriate material such as an inorganic material such as stainless steel or glass, or an organic material such as polystyrene, polyethylene terephthalate copolymer, or polypropylene.

The Labyrinthulomycetes can be cultured under appropriate temperature conditions, pH conditions, aeration conditions, and the like. The culture temperature is preferably 5° C. or higher and 40° C. or lower, more preferably 10° C. or higher and 35° C. or lower, and even more preferably 10° C. or higher and 30° C. or lower. The pH is preferably 2 or higher and 11 or lower, more preferably 4 or higher and 9 or lower, and even more preferably 6 or higher and 8 or lower.

The Labyrinthulomycetes can be cultured while being subcultured at appropriate intervals depending on the genus and of the Labyrinthulomycetes, the medium species composition, the culture conditions, and the like. For example, in the Labyrinthulomycetes, the logarithmic growth phase ends about 2 days after the start of culture, and the death phase begins about 7 days later. Therefore, the subculture of Labyrinthulomycetes is preferably performed at intervals of 1 day or more and 10 days or less, more preferably at intervals of 2 days or more and 7 days or less, and even more preferably at intervals of 2 days or more and 5 days or less. The culture time of the Labyrinthulomycetes can be set to an appropriate time depending on the genus and species of the Labyrinthulomycetes, the medium composition, the culture conditions, the purpose of culture, and the like. In particular, Aurantiochytrium algae, which are Labyrinthulomycetes algae, are preferable because they are heterotrophic algae that live in brackish water areas and have the characteristic of assimilating nutrients in water to produce lipids and accumulate them in cells.

It is preferable to use a strain of Aurantiochytrium algae that has excellent ability to produce desired triglycerides. Such algal strains may be naturally collected and isolated, cloned through mutation induction and screening, or established using genetic recombination technology. For example, Aurantiochytrium Sp. SA-96 strain, NIES-3737 strain, Aurantiochytrium NB6-3 strain, or Aurantiochytrium mh1959 strain have the property of accumulating large amounts of triglycerides containing pentadecanoic acid (PDA), an odd-chain fatty acid, and triglycerides containing docosahexaenoic acid (DHA) and docosapentaenoic acid (DPA), highly unsaturated fatty acids, in cells, and are therefore particularly preferable as microorganisms used for producing the pentadecanoic acid triglycerides of the present technology.

The culture of the Aurantiochytrium algae is carried out by a method established in the technical field. That is, ordinary maintenance culture is performed by inoculating algae into a medium appropriately prepared with components and according to a conventional method. A medium for culturing Aurantiochytrium algae essentially contains salt, a carbon source, and a nitrogen source. Generally, so-called GTY medium (10-40 g/L artificial seawater, 20-100 g/L D (+)-glucose, 10-60 g/L tryptone, 5-40 g/L yeast extract) is used for culturing microalgae.

Examples of the carbon source include sugars such as glucose, fructose, and sucrose. These carbon sources are added at a concentration of, for example, 20 to 120 g per liter of medium.

Aurantiochytrium algae are marine algae, and an appropriate amount of artificial seawater is added to the medium. Preferably, the artificial seawater is added so that the final salt concentration of the medium is about 10% (v/v) to about 100% (v/v) of seawater (salt concentration 3.4% (w/v)), for example, the salt concentration is about 1.0 to 3.0% (w/v).

Generally, various nitrogen sources such as organic nitrogen such as sodium glutamate and urea, or inorganic nitrogen such as ammonium acetate, ammonium sulfate, ammonium chloride, sodium nitrate, and ammonium nitrate, or biologically derived digests such as yeast extract, corn steep liquor, polypeptone, peptone, and tryptone can be added to the culture medium for microalgae. In particular, a cell extract obtained by extracting liquid components from various animal cells is preferably used as a nitrogen source to be added to a medium used for culturing Aurantiochytrium algae. The use of cell extracts, which are rich in nutrients such as amino acids, nucleic acids, vitamins, and minerals derived from cells and are available at low cost, is extremely advantageous when cells must be mass-cultured on an industrial scale to obtain cultured cell products.

However, as described above, when a medium prepared based on a cell extract is used, the ratio of odd-chain fatty acids in the triglycerides produced by the cultured algae is significantly reduced. Therefore, when efficiently producing the target product of the present technology, it was not possible to use a cell extract as a nitrogen source in the medium. Therefore, the present inventors have already reported a method for producing triglycerides containing odd-chain fatty acids as main components (Japanese Patent Application Laid-Open No. 2017-063633), in which Aurantiochytrium algae were cultured in an algae culture medium prepared by adding a cell extract treated with a strong acid, and found that the production amount of odd-chain fatty acids was dramatically increased as compared with the case where a cell extract not subjected to the treatment was added.

Furthermore, in a preferred embodiment of the present technology, the basal medium for culturing Aurantiochytrium algae is prepared by adding 10 to 50 mM valine and 10 to 50 mM sodium propionate to a medium containing 2% or more glucose, 0.5 to 4% sodium glutamate, 0.1 to 2% yeast extract, 1 to 3.3% sea salt, and 2 to 20% whey (animal or vegetable). As the animal or vegetable whey, tofu whey (soybean whey) is preferable. 2% or more of the culture solution of Aurantiochytrium pre-cultured at 20 to 30° C. for 72 hours with 2% or more glucose, 0.5 to 4% sodium glutamate, 0.1 to 2% yeast extract, 1 to 3.3% sea salt, and 2 to 20% whey (animal or vegetable) is added to this basal medium. Air is passed through the Aurantiochytrium-added culture solution, and the solution is gently stirred. The culture is carried out for 48 to 200 hours while maintaining the temperature at 20 to 30° C. and the pH at 5.0 to 8.5 (1.0 M NaOH solution is used for pH adjustment). After the culture, Aurantiochytrium cells that have produced pentadecanoic acid triglycerides can be collected by centrifugation (see WO2020/054804 pamphlet).

The pellet collected from the culture solution obtained by the method as described above by centrifugation or filtration is dried by freeze-drying or heating. Alternatively, the medium in which algal cells after culture are suspended may be used as it is in the triglyceride extraction step. The extraction may be performed multiple times using different organic solvents. As the organic solvent, a mixture of a polar solvent and a weakly polar solvent such as an n-hexane/ethanol mixed solvent, chloroform/methanol mixed solvent, or an ethanol/diethyl ether mixed solvent can be used. The obtained extract is purified by a method known to those skilled in the art.

As a method for separating triglycerides, a fractionation method known to those skilled in the art is adopted. Separation and purification may be performed using various physicochemical properties such as polarity, solubility in a solvent, melting point, specific gravity, and molecular weight of a triglyceride molecule to be fractionated, and column chromatography technology is preferably used. The conditions of the triglyceride separation means can be set by ordinary condition examination by those skilled in the art depending on the composition of the triglyceride mixture and the type of triglyceride to be fractionated.

Algae, Schizochytrium algae and Aurantiochytrium algae can synthesize and accumulate both odd-chain fatty acid triglycerides and highly unsaturated fatty acid triglycerides in cells. Therefore, after adding ethanol, hexane, or ethyl acetate to the obtained algal cells to extract lipids, the solvent is distilled off to obtain algal lipids. By allowing this lipid to stand at 5° C., pentadecanoic acid triglycerides can be precipitated. The composition of the purified pentadecanoic acid triglyceride “PdATG” can be analyzed by HPLC-MS, HPLC, gas chromatography, or the like.

Aurantiochytrium algae can synthesize and accumulate both odd-chain fatty acid triglycerides and highly unsaturated fatty acid triglycerides in cells. Therefore, after adding hexane or ethyl acetate to the obtained Aurantiochytrium cells to extract lipids, hydrogen peroxide water is added to this lipid solution, or ozone is passed through to oxidatively decompose unsaturated fatty acids. After the reaction is completed, the oxides are removed with sodium hydrogen carbonate and sodium carbonate or an ion exchange resin to obtain pentadecanoic acid triglycerides “PdATG”. The composition of the purified pentadecanoic acid triglyceride “PdATG” can be analyzed by HPLC-MS, HPLC, gas chromatography, or the like.

### (Functions and Effects)

The active ingredient of the present technology has an effect of normally caused ameliorating diseases by endoplasmic reticulum stress and poor physical condition before the onset of the diseases by alleviating endoplasmic reticulum stress in ocular tissue cells represented by retinal cells. Furthermore, it is considered that retinal ganglion cell death can be inhibited by alleviating endoplasmic reticulum stress, and the ameliorating effect was also observed for visual field defect disorders and glaucoma.

This means that it is very important to establish a preventive method from a period considerably before the onset. As one of the measures for prevention, it is conceivable to prevent neurodegeneration by ingesting preventive and palliative components from before the onset. At the same time, treatment from an early stage of the initial state suspected of onset is also important.

In the present technology, “light-induced ocular tissue disorder” means various disorders in ocular tissues caused by exposure to light. Typically, it includes retinal inflammation, particularly retinal inflammation caused by exposure to light of a specific wavelength, but is not particularly limited to retinal inflammation and includes disorders in ocular tissues in a broad sense. The light of a specific wavelength refers to light having a wavelength of 10 to 830 nm, particularly so-called blue light. Blue light refers to light having a wavelength of 380 to 530 nm, particularly light having a wavelength of 380 to 495 nm, and mainly refers to light emitted from IT devices such as personal computers and smartphones, and light emitted from LEDs. Cell death of photoreceptor cells is caused by irradiation with such specific light, and endoplasmic reticulum stress, particularly increased expression of Activating Transcriptional Factor 4 (ATF4), a downstream factor of the PERK pathway, is involved in the mechanism.

In the present technology, “disorders related to light-induced ocular tissue disorders” means systemic symptoms affected by various disorders in ocular tissues caused by exposure to light and visual function decline. This includes a state of suffering from various diseases, or an uncomfortable state of the eyes that cannot be called a disease. Here, the “uncomfortable state of the eyes” includes a state where the eyes are objectively or subjectively tired or dry. Subjectively, in a state where the eyes are tired, not only sensations about the eyes but also sensations about other parts of the body may be accompanied, for example, sensations such as eye pain, blurred vision, tearing, stiff shoulders and lower back, eye fatigue, flickering vision, double vision, headache, irritability, foreign body sensation in the eyes, hyperemia of the eyes, dazzling light, decreased concentration, and discomfort due to eye symptoms may be accompanied. It should be noted that the present technology is not limited to these exemplified symptoms, and includes any disorder or discomfort caused by ocular tissue disorders.

### (Prophylactic for Light-Induced Ocular Tissue Disorders and Related Disorders)

The prophylactic for light-induced ocular tissue disorders and related disorders according to the first aspect of the present technology includes a preparation that inhibits ocular tissue cell death due to abnormal protein accumulation in the endoplasmic reticulum. The ocular tissue cell death herein includes necrosis and apoptosis of ocular tissue cells.

### (Progression Inhibitor and Ameliorant for Light-Induced Ocular Tissue Disorders and Related Disorders)

The progression inhibitor for light-induced ocular tissue disorders and related disorders according to the second aspect of the present technology and the ameliorant for light-induced ocular tissue disorders and related disorders according to the third aspect of the present technology contain the pentadecanoic acid triglyceride represented by the above formula (I) as an active ingredient, can be used to ameliorate the symptoms of the light-induced ocular tissue disorders and related disorders described above, and are useful as pharmaceuticals therefor.

Here, the term “inhibition of ocular tissue cell death” or “inhibition of retinal cell death” includes alleviating, reducing, or eliminating ocular tissue cell death or retinal cell death, inhibiting the progression of ocular tissue cell death or retinal cell death, and preventing and inhibiting it. It can be used for the prevention, progression inhibition, and/or amelioration of the disorders (diseases and discomfort) as described above that are manifested by ocular tissue cell death or retinal cell death caused by endoplasmic reticulum stress.

### (Pharmaceuticals)

The term “pharmaceuticals” means a therapeutic agent for preventing, inhibiting the progression, and/or ameliorating the symptoms of light-induced ocular tissue disorders and related disorders by inhibiting endoplasmic reticulum stress in patients with light-induced ocular tissue disorders and related disorders.

The composition or pharmaceuticals according to the present technology is effective not only in humans but also in mammals including domestic animals such as cattle, horses, pigs, and goats, and pet animals such as dogs and cats.

The composition or pharmaceuticals according to the present technology may contain only the compound of formula (I) as an active ingredient, or may contain other components as long as they do not inhibit the ocular tissue cell death inhibitory effect. Other components may be, for example, conventionally used therapeutic agents or prophylactic agents for ocular tissue diseases. Therefore, the endoplasmic reticulum stress inhibitor of the present embodiment provides a pharmaceutical composition for preventing and ameliorating ocular tissue degenerative diseases in a further embodiment.

The composition or pharmaceuticals according to the present technology can be orally administered, and can be prepared in the form of granules, powders, tablets (including sugar-coated tablets), pills, capsules, syrups, emulsions, suspensions, and the like as dosage forms suitable for oral administration. These preparations can be formulated using pharmaceutically acceptable carriers by methods commonly used in the art. Examples of pharmaceutically acceptable carriers include excipients, binders, diluents, additives, fragrances, buffers, thickeners, colorants, stabilizers, emulsifiers, dispersants, suspending agents, preservatives, and the like.

Although not particularly limited, more specifically, for example, when producing a pharmaceutical product by blending the pentadecanoic acid triglyceride represented by the above formula (I), for example, sugars such as dextrin and starch; proteins such as gelatin, soy protein, and corn protein; amino acids such as alanine, glutamine, and isoleucine; polysaccharides such as cellulose and gum arabic; and any auxiliary agents such as oils and fats such as soybean oil and medium-chain fatty acid triglycerides can be added to formulate into any dosage form.

The blending amount of the pentadecanoic acid triglyceride represented by the above formula (I) in the pharmaceuticals according to the present technology is not particularly limited, but it is preferable to adjust the intake of pentadecanoic acid triglyceride per adult per day, which is a concentration exhibiting effectiveness, to be about 10 to 1000 mg per day.

The composition or pharmaceuticals according to the present technology is not limited to an oral administration form, but may be a parenteral administration form, and may be, for example, a suspension eye drop, an ophthalmic ointment, an injection, or an infusion. In this case, it can be formulated using pharmaceutically acceptable auxiliary agents, carriers, and the like by methods commonly used in the art.

### (Food Products)

The food product according to the fourth aspect of the present technology contains the pentadecanoic acid triglyceride represented by the above formula (I) as an active ingredient, can be ingested over a long period of time as a preventive food and drink from before the onset of light-induced ocular tissue disorders and related disorders, and is useful as a health food therefor. It has been reported that pentadecanoic acid constituting PdATG is contained in small amounts in edible parts such as meat of cows, pigs, chickens, and sheep, fish living in rivers and seas, and mushrooms, and it is inferred that PdATG is also contained in trace amounts and has high safety from many years of eating experience.

Therefore, the food product of the present embodiment is useful as a health food taken for health promotion. Here, the “health food” means food and drink intended to be used for preventing, inhibiting the progression, alleviating or ameliorating the above-mentioned light-induced ocular tissue disorders or related disorders such as eye fatigue and blurred vision, or, in addition to this, for preventing, inhibiting the progression, alleviating or ameliorating forgetfulness due to daily promotion and aging, decline in understanding and judgment, memory impairment, disorientation, executive function disorder, aphasia/apraxia/agnosia, and dementia, and refers to a broad sense of “health food” including foods with function claims, foods with nutrient function claims, and foods for specified health use under the “Foods with Health Claims System” that satisfy the standards for safety and effectiveness established by the government.

When producing a food product by blending the pentadecanoic acid triglyceride represented by the above formula (I), any auxiliary agents such as sugars such as dextrin and starch; proteins such as gelatin, soy protein, and corn protein; amino acids such as alanine, glutamine, and isoleucine; polysaccharides such as cellulose and gum arabic; and oils and fats such as soybean oil and medium-chain fatty acid triglycerides can be added to formulate into any dosage form.

The blending amount of the pentadecanoic acid triglyceride represented by the above formula (I) in the food product of the present technology is not particularly limited, but it is preferable to adjust the intake of pentadecanoic acid triglyceride per adult per day to be about 1 to 100 mg, taking into account the general intake of the food to be added.

Specific examples of the food product include, for example, beverages such as soft drinks, carbonated drinks, nutritional drinks, fruit drinks, and lactic acid drinks (including concentrated liquids and powders for preparation of these beverages); frozen desserts such as ice cream, ice sherbet, and shaved ice; noodles such as soba, udon, harusame, gyoza skins, shumai skins, Chinese noodles, and instant noodles; confectionery such as candy, gum, chocolate, snacks, biscuits, jelly, jam, cream, and baked goods; seafood and livestock processed foods such as kamaboko, ham, and sausages; dairy products such as processed milk and fermented milk; oils and fats and processed oil and fat foods such as salad oil, tempura oil, margarine, mayonnaise, shortening, whipped cream, and dressings; seasonings such as sauces and dips; health and nutritional supplements in various forms such as tablets and granules; and other examples include soups, stews, salads, side dishes, and pickles.

The food product according to the present technology may contain various food additives, for example, antioxidants, fragrances, various esters, organic acids, organic acid salts, inorganic acids, inorganic acid salts, inorganic salts, colorants, emulsifiers, preservatives, seasonings, sweeteners, acidulants, fruit juice extracts, vegetable extracts, nectar extracts, pH adjusters, and quality stabilizers, alone or in combination.

The content concentration of pentadecanoic acid triglyceride in the food product according to the present technology is about 0.00001 to 100% by mass (hereinafter represented by %), preferably about 0.0005 to 50% by mass as a solid content, which provides usability and good effects.

Specific examples of the food product include, but are not limited to, food products for improving light-induced ocular tissue damage and related disorders, food products for improving glaucoma and retinitis pigmentosa, food products for relieving eye strain, shoulder and lower back stiffness, irritability, headache, and other discomforts caused by eye fatigue.

Preventive agent for visual field defect disorders, glaucoma, and related disorders) The preventive agent for visual field defect disorders, glaucoma, and related disorders according to the fifth aspect of the present technology contains the pentadecanoic acid triglyceride represented by the above formula (I) as an active ingredient.

### (Progression Inhibitors and Ameliorating Agents for Visual Field Defect Disorders, Glaucoma, and Related Disorders)

The progression inhibitor for visual field defect disorders, glaucoma, and related disorders according to the sixth aspect of the present technology and the ameliorating agent for visual field defect disorders, glaucoma, and related disorders according to the seventh aspect of the present technology contain the pentadecanoic acid triglyceride represented by the above formula (I) as an active ingredient, and can be used to ameliorate the symptoms of the visual field defect disorders, glaucoma, and related disorders described above, and are useful as pharmaceuticals for that purpose.

The detailed description of the preventive agent for visual field defect disorders, glaucoma, and related disorders according to the fifth aspect, the progression inhibitor for visual field defect disorders, glaucoma, and related disorders according to the sixth aspect, and the ameliorating agent for visual field defect disorders, glaucoma, and related disorders according to the seventh aspect, or these pharmaceuticals, is almost the same as the description of the preventive agent for light-induced ocular tissue damage and related disorders according to the first aspect, the progression inhibitor for light-induced ocular tissue damage and related disorders according to the second aspect, and the ameliorating agent for light-induced ocular tissue damage and related disorders according to the third aspect, or these pharmaceuticals, and is omitted to avoid duplication. The food product according to the fourth aspect can also be expected to have preventive effects against visual field defect disorders, glaucoma, and related disorders.

Next, the present technology will be described in more detail with reference to examples, but the present technology is not limited to these examples. In the following examples, the unit % of the numerical values indicating the addition amount of various components means % by mass.

### EXAMPLES

### Production Example 1

### Production of Pentadecanoic Acid Triglyceride Using Aurantiochytrium Aurantiochytrium

Aurantiochytrium Aurantiochytrium mh1959 strain (purchased from Professor Masahiro Hayashi, Faculty of Agriculture, Miyazaki University, National University Corporation) was pre-cultured at 25° C. for 72 hours using a medium containing 3.6% glucose, 0.5% sodium glutamate, 0.2% yeast extract, 1% sea salt, and 10% whey. This was added to the following basic medium at 2%, and air was passed through and gently stirred. The basic medium 1 kg was prepared by adding 50 mM valine and 25 mM sodium propionate to a medium containing 3.6% glucose, 0.5% sodium glutamate, 0.2% yeast extract, 1% sea salt, and 10% whey. The culture was carried out at 25° C., pH was maintained at 7.40 to 7.75 (1.0 M NaOH solution was used for pH adjustment), and cultured for 72 to 96 hours.

After culturing, the cells were centrifuged at 3000 rpm for 15 minutes, and about 20 g of algal cells were collected. Hexane or ethyl acetate was added to 20 g of the obtained Aurantiochytrium algal cells, and lipids were extracted. Hydrogen peroxide solution was added to the extracted lipid solution (water was added as necessary), and ozone was passed through at room temperature. After the reaction was completed, the oxides were removed with sodium bicarbonate and sodium carbonate or an ion exchange resin, and 2 g of a pentadecanoic acid triglyceride mixture that precipitated as the temperature decreased was obtained.

### (Composition Analysis of Pentadecanoic Acid Triglyceride)

TO the lipid containing the pentadecanoic acid triglyceride obtained in Production Example 1, 0.50 mL of 14% BF3-methanol and 0.25 mL of methyl acetate were added, and the mixture was heated at 70° C. for 30 minutes to obtain a methyl ester of fatty acid (FAME). Exactly 1.0 mL of n-hexane and 5 mL of physiological saline were added to the reaction solution, and the mixture was vigorously mixed. The mixture was centrifuged at 2800 rpm for 10 minutes, and the n-hexane layer was used as a sample for gas chromatography.

The sample was analyzed using a gas chromatograph device GC-2025 manufactured by Shimadzu Corporation. The analysis conditions were as follows: Agilent J&W GC column DB-23 (30 m×0.25 mm) was used, 1 μL of the sample was injected, and detected by FID (flame ionization detector) with a carrier gas (He, 14 psi). The molecular species of FAME was identified based on the retention time of the fatty acid methyl ester standard product (manufactured by GL Sciences Inc.). The fatty acid composition was determined from the area ratio. The determined composition is a mass ratio. The ratio of odd-chain fatty acids was determined by multiplying the total fatty acid amount by the ratio (%) of odd-chain fatty acids (C13, C15, C17). The results obtained are shown in Table 1 below.

TABLE 1

| Fatty | Retention | Peak | Ratio |
|---|---|---|---|
| acid | time (min) | area | (mass %) |
| C11:0 | 7.939 | 2207 | 0.3 |
| C12:0 | 8.483 | 2735 | 0.3 |
| C13:0 | 9.065 | 39249 | 5.3 |
| C14:0 | 9.715 | 99919 | 13.4 |
| C15:0 | 10.461 | 422015 | 56.7 |
| C16:0 | 11.291 | 134108 | 18.0 |
| C17:0 | 12.23 | 44681 | 6.0 |

From the results shown in Table 1, the content of odd-chain fatty acids in the triglyceride obtained in Production Example 1 was 68.3% by mass ratio. The fatty acids that make up the triglycerides were found to be mainly pentadecanoic acid residues (C15) and palmitic acid residues (C16).

### (Mass Analysis of Pentadecanoic Acid Triglyceride)

Lipids containing pentadecanoic acid triglyceride obtained in Production Example 1 were analyzed by mass spectrometry using a Thermo Fischer Orbitrap mass spectrometer Exactive Plus (AMR DART ion source).

As a result, the fragment composition of the major mass spectral peaks indicated that the pentadecanoic acid triglyceride obtained in Production Example 1 is a triglyceride mixture containing mainly a triglyceride formed only with pentadecanoic acid residues (C15) and another triglyceride containing two units of pentadecanoic acid residues (C15) and one unit of palmitic acid residue (C16).

### (Example 1) Study of Cell Death Inhibitory Effect by Reducing Endoplasmic Reticulum Stress in Retinal Cells by PdATG

In order to examine the effect of PdATG, the pentadecanoic acid triglyceride obtained in Production Example 1 was used as a test substance, and the following experiment was conducted.

### Test Method

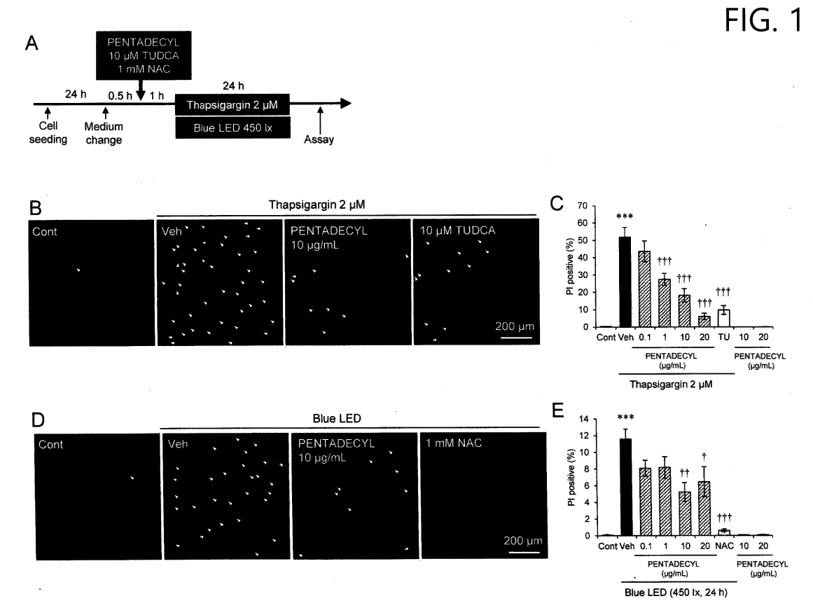

Cells: Mouse-derived 661W cone photoreceptor cell line was used. Cells were seeded in a 96-well plate at a density of 3,000 cells and cultured for 24 hours at 37° C. under 5% CO: in DMEM medium containing 10% fetal bovine serum (FBS), penicillin (100 U/ml) and streptomycin (100 μg/ml). After that, the medium was replaced with 1% FBS-containing DMEM medium and incubated at 37° C. for 30 minutes. PdATG obtained in Production Example 1 was added at concentrations of 0.1, 1, 10, or 20 μm/mL, or 10 UM tauro-ursodeoxycholic acid (TUDCA) and 1 mM N-acetylcysteine (NAC) were added. After 1 hour of culture, endoplasmic reticulum stress was induced by adding 2 μM thapsigargin, or light damage was induced by irradiation with blue LED light at 450 1×. 24 hours after the damage, the dead cell rate was evaluated by Hoechst & PI staining. That is, Hoechst 33342 (8.1 UM) and Propidium Iodide (PI) (1.5 μM) were added, and after nuclear staining by incubation at 37° C. for 15 minutes, images were taken with a fluorescence microscope (DP30BW; Olympus). Hoechst 33342 (λex=360 nm; λem>490 nm) that stained cell nuclei was defined as all cells, PI (λex=535 nm; λem>617 nm) that stained dead cells was counted, and the dead cell rate was calculated. The results obtained are shown in FIG. 1. Regarding statistical analysis, all experimental results are expressed as mean±standard error. Statistical analysis was performed using Tukey's test or Dunnett's test.

As shown in FIG. 1, the dead cell rate increased with the addition of thapsigargin, and was suppressed in a concentration-dependent manner at concentrations of 1 μm/mL to 20 μm/mL with the addition of PdATG (see FIGS. 1B and C). In blue LED light irradiation, the increase in dead cell rate was suppressed at a concentration of 10 μm/mL (see FIGS. 1D and E).

### (Example 2) Study of the Inhibitory Effect of PdATG on the Expression of Endoplasmic Reticulum Stress-Related Proteins in Retinal Cells

In order to examine the effect of PdATG, the pentadecanoic acid triglyceride obtained in Production Example 1 was used as a test substance, and the following experiment was conducted.

### Test Method

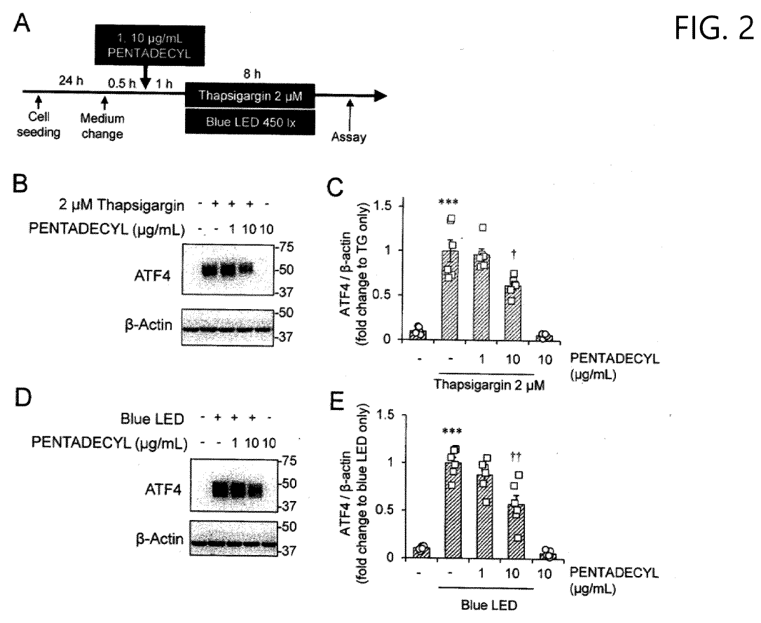

Cells: Mouse-derived 661W cone photoreceptor cell line was used. Cells were seeded in a 12-well plate at a density of 25,000 cells and cultured for 24 hours at 37° C. under 5% CO2 in DMEM medium containing 10% fetal bovine serum (FBS), penicillin (100 U/ml) and streptomycin (100 μg/ml). After that, the medium was replaced with 1% FBS-containing DMEM medium and incubated at 37° C. for 30 minutes. PdATG obtained in Production Example 1 was added at a concentration of 1 or 10 μm/mL. After 1 hour of culture, endoplasmic reticulum stress was induced by adding 2 μM thapsigargin, or light damage was induced by irradiation with blue LED light at 450 1×. 8 hours after the damage, the expression level of ATF4, an endoplasmic reticulum stress marker, was evaluated by Western blotting. That is, sampling was performed 8 hours after the damage, cells were collected, washed with phosphate buffered saline (1×PBS; 136.9 mM NaCl, 2.68 mM KCl, 10.14 mM Na₂HPO₄·12H₂O, 1.76 mM KH₂PO₄, pH 7.3), and then cell lysate (RIPA buffer) containing 18 protease inhibitor cocktail and phosphatase inhibitor cocktail 2/3 (Sigma-Aldrich) was added, and cell extract was collected. The protein concentration of the cell extract was quantified using a BCA Protein Assay Kit (Thermo Fisher Scientific Inc.), and then suspended in a 10% 2-mercaptoethanol-containing sample buffer so that the protein concentration was uniform, and boiled for 5 minutes. After that, electrophoresis was performed using a 5-20% polyacrylamide gel (SuperSep (trademark)). After applying the sample to the polyacrylamide gel, electrophoresis was performed at 20 mA per gel for 90 minutes. Proteins were separated by differences in molecular weight, and the separated proteins were transferred to a polyvinylidene difluoride (PVDF) membrane (Immobilon-P; Millipore Corporation, Billerica, MA, USA). After blocking the PVDF membrane after transfer with Blocking One-P, it was immersed in a primary antibody diluted with Can get signal solution 1 and reacted overnight at 4° C. After that, the transfer membrane was washed with 50 mM TBS containing 0.05% Tween 20 (T-TBS: 10 mM Tris, 40 mM Tris hydrochloride, 150 mM NaCl), immersed in a secondary antibody diluted with Can get signal solution 2, and reacted at room temperature for 1 hour. After washing with T-TBS, the bands of the immunoreaction were detected with a fluorescent substrate (ImmunoStar LD; Wako Pure Chemical Industries, Ltd.). The density of the detected bands was imaged with LAS-4000 mini (Fujifilm), and the amount of each protein was quantified by analyzing with gel analysis software (Image Reader LAS-4000; Fujifilm) and detected band analysis software (Multi Gauge; Fujifilm). The band intensity of each protein was corrected using β-actin. The primary antibodies used were mouse anti-GRP78/BiP (1:500; Becton Dickinson Company), rabbit anti-GRP94, rabbit anti-ATF4, mouse anti-ubiquitin (1:1,000; Cell Signaling Technology), and mouse anti-β-actin (1:5,000; Sigma-Aldrich). The secondary antibodies used were horseradish peroxidase (HRP)-conjugated goat anti-rabbit or goat anti-mouse (1:2,000; Thermo Fisher Scientific Inc.). Regarding statistical analysis, all experimental results are expressed as mean±standard error. Statistical analysis was performed using Tukey's test or Dunnett's test. The results obtained are shown in FIG. 2.

As shown in FIG. 2, the expression level of ATF4 increased due to endoplasmic reticulum stress caused by the addition of thapsigargin, and PdATG significantly decreased the expression level at a concentration of 10 μm/mL (see FIGS. 2B and C). A decrease in expression level was also observed at 10 μm/mL in light damage caused by blue LED light.

### (Example 3) Study of the Inhibitory Effect of PdATG on the Expression of Endoplasmic Reticulum Stress-Related mRNA in Retinal Cells

In order to examine the effect of PdATG, the pentadecanoic acid triglyceride obtained in Production Example 1 was used as a test substance, and the following experiment was conducted.

### Test Method

Cells: Mouse-derived 661W cone photoreceptor cell line was used. Cells were seeded at 3×104 cells/well in a 12-well plate and cultured for 24 hours at 37° C. under 5% CO2 in DMEM medium containing 10% fetal bovine serum (FBS), penicillin (100 U/ml) and streptomycin (100 μg/ml). After that, the medium was replaced with 1% FBS-containing DMEM medium and incubated at 37° C. for 30 minutes. PdATG obtained in Production Example 1 was added at concentrations of 0.1, 1, 10, 20, or 50 μm/mL. After 1 hour of culture, endoplasmic reticulum stress was induced by adding 2 μM thapsigargin. 8 hours after the damage, the mRNA amount was evaluated by RT-PCR. For RT-PCR, cells were collected by sampling, and RNA was extracted using Nucleo Spin RNA II (Takara Bio Inc.).

As a result, the expression levels of endoplasmic reticulum stress-related mRNAs, which were increased by the addition of thapsigargin, were suppressed by the addition of pentadecyl, Arf4, Bip, and Grp94.

### (Example 4) Effect of PdATG-Containing Test Food on Glaucoma Patients

### Test Method

Aurantiochytrium mh1959 strain was cultured in the same manner as in Example 1, and hexane was added to the obtained Aurantiochytrium algal cells to extract lipids. Then, the obtained lipids were filled into soft capsules (manufactured by Byopharma Co., Ltd.) as a shape that is easy to take, and used as test food. Table 2 shows the raw material composition per capsule of the test food. Each capsule contained 6 mg of PdATG.

TABLE 2
Test food composition

| | | Volume |
|---|---|---|
| Contents | Aurantiochytrium- | 24 mg (6 mg) |
| | derived oil (PdATG) | |
| | Edible corn oil | 126 mg |
| Soft capsule | Gelatin, Glycerin, | Small |
| coating | Glycerin fatty | quantity |
| | acid ester, | |

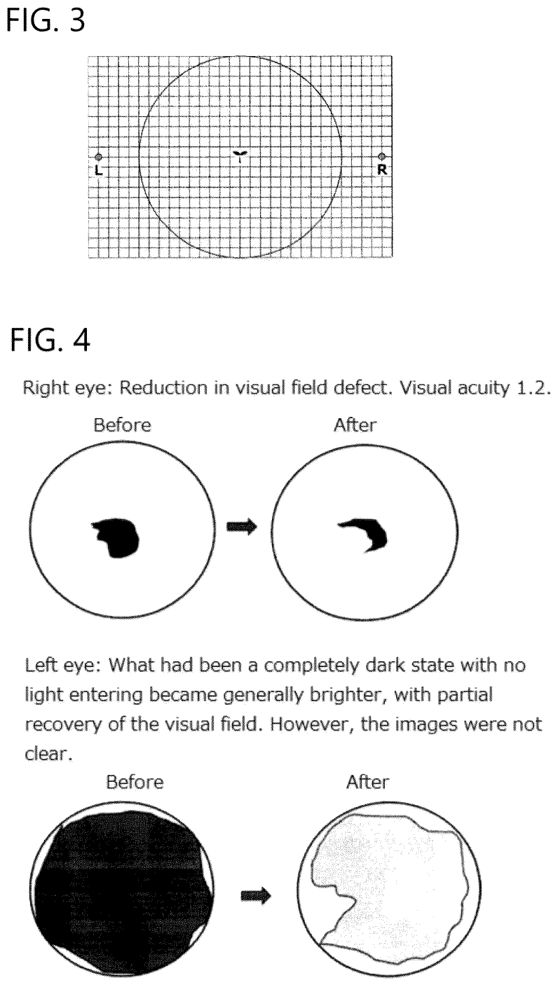

The subject was a 67-year-old male with glaucoma, whose condition was that the right eye had almost lost its field of vision, and the left eye had a visual field defect in the center of the eye. The dosage was 2 capsules per day for 6 months, and then 8 capsules per day for 3 months. Evaluation by visual field check was evaluated by a questionnaire and hearing filled out by the patient. <Visual field check> Close one eye, fixate on the double leaves in the center of the chart shown in FIG. 3 from a position about 30 cm in front, and check for “there is a blind spot in the field of vision”, “partially dark”, and “grid is distorted”.

As a result, as shown in FIG. 4, partial recovery of the constricted visual field and partial visual acuity were recovered. That is, the visual field defect in the right eye decreased and visual acuity became 1.2, and the left eye, which was in a state where black light did not enter the whole, became brighter as a whole, and the visual field was partially restored.

### (Example 5) Effect of PdATG-Containing Eye Drops on Glaucoma Patients

### Test Method

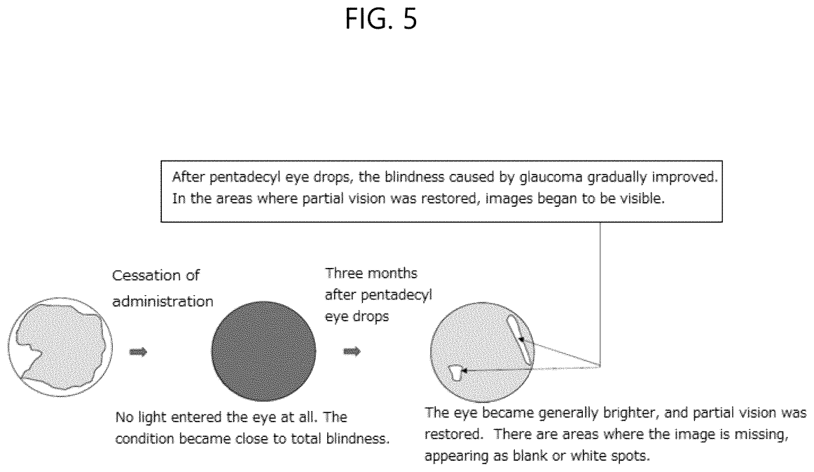

Hexane was added to Aurantiochytrium cells obtained by culturing Aurantiochytrium algae in the same manner as in Production Example 1 to extract lipids, and the extracted lipids were cooled and crystallized to obtain a pentadecanoic acid triglyceride mixture. 1 mg of this pentadecanoic acid triglyceride mixture was dissolved in 100 mL of contact tear fluid (containing 5.5 mg of sodium chloride, 1.5 mg of potassium chloride, 0.05 mg of glucose, and 1 mg of aminoethylsulfonic acid in 1 ml, and trace amounts of boric acid, borax, hypromellose, polyoxyethylene hydrogenated castor oil 60, alkyldiaminoethylglycine hydrochloride, 1-menthol, and pH adjuster as additives) while warming to about 60° C., and after sterile filtration, pentadecyl-containing eye drops were prepared. The subject was the same 67-year-old male who took the test food in Example 4. After taking the test food, when the administration was temporarily discontinued, the visual field constriction of the left eye, which is a symptom of glaucoma, worsened and became almost blind. Therefore, the pentadecyl-containing eye drops prepared above were instilled for 3 months. As a result, as shown in FIG. 5, the blindness state in which black light did not enter the whole gradually recovered, and the whole became brighter, and visual acuity partially recovered, and images began to be seen.

1-15. (canceled)

16. A method of preventing light-induced ocular tissue damage and related disorders, comprising:
administering a triglyceride represented by the following formula (I) to a subject in need thereof: 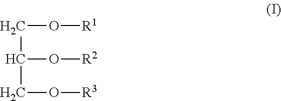
wherein R¹, R², and R³ are each a saturated fatty acid residue, and at least one of them is a pentadecanoic acid residue.

17. The method according to claim 16, wherein R¹ and R² or R¹ and R³ in formula (I) are pentadecanoic acid residues.

18. The method according to claim 16, wherein any one of R¹, R², and R³ in formula (I) is tridecylic acid (C13), myristic acid residue (C14), palmitic acid residue (C16), or margaric acid residue (C17).

19. The method according to claim 16,
wherein in the triglyceride, all of R¹, R², and R³ in formula (I) are pentadecanoic acid residues, and
wherein the method further comprises administering a therapeutically-effective amount of a second triglyceride according to formula (I) to the subject, wherein, in the second triglyceride, any two of R¹, R², and R³ in formula (I) are pentadecanoic acid residues and the other one is a myristic acid or palmitic acid residue.

20. The method according to claim 16, wherein the triglyceride of formula (I) is derived from algae of the genus Aurantiochytrium or the genus Schizochytrium.

21. A method of ameliorating or inhibiting progression of light-induced ocular tissue damage and related disorders, comprising:
administering a triglyceride represented by the following formula (I) to a subject in need thereof: 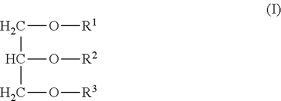
wherein R¹, R², and R³ are each a saturated fatty acid residue, and at least one of them is a pentadecanoic acid residue.

22. The method according to claim 21, wherein R¹ and R² or R¹ and R³ in formula (I) are pentadecanoic acid residues.

23. The method according to claim 21, wherein any one of R¹, R², and R³ in formula (I) is tridecylic acid (C13), myristic acid residue (C14), palmitic acid residue (C16), or margaric acid residue (C17).

24. The method according to claim 21,
wherein in the triglyceride, all of R¹, R², and R³ in formula (I) are pentadecanoic acid residues, and
wherein the method further comprises administering a second triglyceride according to formula (I) to the subject, wherein, in the second triglyceride, any two of R¹, R², and R³ in formula (I) are pentadecanoic acid residues and the other one is a myristic acid or palmitic acid residue.

25. The method according to claim 21, wherein the triglyceride of formula (I) is derived from algae of the genus Aurantiochytrium or the genus Schizochytrium.

26. A method of preventing visual field defect disorders, glaucoma, and related disorders, comprising:
administering a triglyceride represented by the following formula (I) to a subject in need thereof: 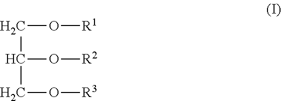
wherein R¹, R², and R³ are each a saturated fatty acid residue, and at least one of them is a pentadecanoic acid residue.

27. The method according to claim 26, wherein R¹ and R² or R¹ and R³ in formula (I) are pentadecanoic acid residues.

28. The method according to claim 26, wherein any one of R¹, R², and R³ in formula (I) is tridecylic acid (C13), myristic acid residue (C14), palmitic acid residue (C16), or margaric acid residue (C17).

29. The method according to claim 26,
wherein in the triglyceride, all of R¹, R², and R³ in formula (I) are pentadecanoic acid residues, and
wherein the method further comprises administering a second triglyceride according to formula (I) to the subject, wherein, in the second triglyceride, any two of R¹, R², and R³ in formula (I) are pentadecanoic acid residues and the other one is a myristic acid or palmitic acid residue.

30. The method according to claim 26, wherein the triglyceride of formula (I) is derived from algae of the genus Aurantiochytrium or the genus Schizochytrium.

31. A method of ameliorating or inhibiting progression of visual field defect disorders, glaucoma, and related disorders, comprising:
administering a triglyceride represented by the following formula (I) to a subject in need thereof: 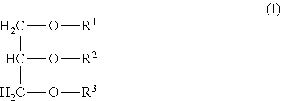
wherein R¹, R², and R³ are each a saturated fatty acid residue, and at least one of them is a pentadecanoic acid residue.

32. The method according to claim 31, wherein R¹ and R² or R¹ and R³ in formula (I) are pentadecanoic acid residues.

33. The method according to claim 31, wherein any one of R¹, R², and R³ in formula (I) is tridecylic acid (C13), myristic acid residue (C14), palmitic acid residue (C16), or margaric acid residue (C17).

34. The method according to claim 31,
wherein in the triglyceride, all of R¹, R², and R³ in formula (I) are pentadecanoic acid residues, and
wherein the method further comprises administering a second triglyceride according to formula (I) to the subject, wherein, in the second triglyceride, any two of R¹, R², and R³ in formula (I) are pentadecanoic acid residues and the other one is a myristic acid or palmitic acid residue.

35. The method according to claim 31, wherein the triglyceride of formula (I) is derived from algae of the genus Aurantiochytrium or the genus Schizochytrium.
